(19) United States
(12) Patent Application Publication
(10) Pub. No.: US 2024/0156104 A1
(43) Pub. Date: May 16, 2024

(54) FUNGI-DERIVED COMPOSITION, ITS PRODUCTION PROCESS AND USES

(71) Applicants: NATURAL RESOURCES INSTITUTE FINLAND (LUKE), Helsinki (FI); UNIVERSITY OF JYVÄSKYLÄ, Jyväskylän yliopisto (FI)

(72) Inventors: Riikka LINNAKOSKI, Helsinki (FI); Varpu MARJOMÄKI, Jyväskylä (FI); Petri KILPELÄINEN, Vantaa (FI); Dhanik RESHAMWALA, Jyväskylä (FI); Henri VANHANEN, Ylämylly (FI); Pyry VETELI, Helsinki (FI)

(21) Appl. No.: 18/547,141

(22) PCT Filed: Feb. 18, 2022
(86) PCT No.: PCT/FI2022/050108

§ 371 Date: Aug. 18, 2023

(30) Foreign Application Priority Data
Feb. 19, 2021 (FI) 20215181

(51) Int. Cl. A01N 65/00; A01N 61/00; A01P 1/00; A61K 8/9728; A61K 36/074; A61L 2/00; A61L 2/18; A61Q 19/00; C12N 1/14; C12P 1/02

(52) U.S. Cl. CPC A01N 65/00; A01N 61/00; A01P 1/00; A61K 8/9728; A61K 36/074; A61L 2/0088; A61L 2/18; A61Q 19/00; C12N 1/145; C12P 1/02; A61K 2800/10; C12R 2001/645

(57) ABSTRACT

A virucidal composition, a method of its manufacturing, related uses and end-user products are hereby provided. The composition includes a mixture of biologically active substances obtainable from selected fungal strains of Ganoderma lucidum (MUS12, MUS15, MUS18 and MUS23) by culturing the fungi in essentially liquid culture medium in a culture tank or a bioreactor in conditions essentially excluding agitation. The biologically active substances are obtained as a product excreted by mycelium of the above indicated G. lucidum strains and contain secondary metabolites, such as ganoderic acids and their derivatives. The composition has proved efficient in preventing the spread of viral infections. The composition can be used as a disinfecting agent for skin and nonliving surfaces and/or as an ingredient in preparation of personal care products.

### FIELD OF THE INVENTION

The present invention generally relates to compositions and methods involving substances derived from Ganoderma lucidum fungi. In particular, the invention concerns a composition comprising a mixture of biologically active compounds obtainable from the selected G. lucidum strains, a method for producing said composition and its uses, in particular, in prevention of spreading viral infections, in disinfection and in production of personal care products.

### BACKGROUND

Viral infections are amongst the most common diseases affecting people worldwide. Combatting against viral infections is constantly challenged with emergence of new serotypes in virus groups due to high mutation rates and low fidelity for viral replication.

The non-enveloped viruses, such as enteroviruses, rotaviruses and norovirus, are stable and remain infectious after staying on surfaces for several weeks and even months. Those viruses show little sensitivity to chemical disinfectants and at present there is no non-toxic means of decontamination of premises infected with mentioned pathogens. The enveloped viruses, such as virus Zika and coronaviruses, including SARS-CoV2—a causative agent for Covid19 pandemics, are generally less stable and more prone to degradation when treated with certain disinfectants. Nevertheless, any one of these viral species is capable of causing serious outbreaks worldwide leading to difficult symptoms and high mortality rates and causing significant losses in global economy.

By way of example, enteroviruses (EVs) represent the world most prevalent viruses. They cause a number of infectious illnesses ranging from mild to severe, including acute infections such as poliomyelitis, hand, foot and mouth disease (HFMD), meningitis, myocarditis, encephalitis and acute flaccid paralysis (AFP). In addition, enteroviruses are an environmental factor in the development of chronic diseases such as type 1 diabetes (TID), asthma and allergies. There are no widely available vaccines against enteroviruses, except for polio, and development of vaccines is complicated by the fact that new enterovirus serotypes emerge constantly.

Overall, development of vaccines lags behind the rates with which new viruses emerge. Additional or alternative ways for preventing the spread of viral infections include disinfecting hands and surfaces, as well as wearing protection gear designed to meet the requirements set by different biosafety levels.

On the other hand, fungi present a vast source of bioactive molecules, while some fungal species contain and/or excrete compounds with reported antiviral activities. In particular, basidiomycetes Ganoderma, in particular G. lucidum, also referred to as Lingzhi- or Reishi mushroom, are widely used for medical purposes in East Asia. Ganoderma species contain triterpenoids, steroids, alkaloids and polysaccharides, with triterpenoids being the most abundant class of compounds obtained from it.

For G. lucidum triterpenoids (more than 150 compounds), various therapeutic effects, such as anti-inflammatory, antioxidant, antihistamine and cholesterol lowering effects, are reported in US 2013/184244 A1 (Minto et al), for example. Medicinal properties of the extracts derived from G. lucidum are further mentioned in U.S. Pat. No. 6,726,911 B (Jüdlich et al). JP 2016-044155A (Takeshi et al) discusses antiviral activity of G. lucidum triterpenoids against influenza virus. In the latter document a mixture of triterpenoid compounds, including various ganoderic acids, has been extracted from fungi with organic solvent, such as chloroform, or by hot water extraction.

Nevertheless, in many instances, screening of wide variety of isolates within the same species is often indispensable to determine whether selected strains do possess expected biological activity. Moreover, industrial scale production of mycelial fungi requires careful optimization of liquid culture conditions, as well as strain selection in order to avoid overproduction of the desired compounds.

Yearly influenza epidemics with new serotypes, the coronavirus pandemic and recent infectious disease outbreaks, such as Ebola and Chikungunya, fuel the demand for efficient surface disinfectant demand. As many viruses commonly spread by direct and indirect contact by coming into contact with secretions from an infected individual including those that carry the infectious agent asymptomatically, there is undeniably a need for safe and broadly acting biocides to encounter viral infections.

In light of abovesaid, it appears desirable to complement and update the field of technology related to developing new, reliable and non-toxic antiviral compounds, in particular, those for external use. In that sense, Ganoderma species provide an attractive target for further exploration of biological potential of said fungi and the substances produced by it.

### SUMMARY OF THE INVENTION

An objective of the present invention is to at least alleviate each of the problems arising from the limitations and disadvantages of the related art. The objective is achieved by various embodiments of a virucidal composition, its production process and related uses.

Thereby, in one aspect of the invention a virucidal composition is provided, according to what is defined in the independent claim 1.

In embodiment, the virucidal composition comprises a mixture of biologically active compounds obtainable from selected strains of Ganoderma lucidum by culturing said fungi in essentially liquid culture medium in a culture tank or a bioreactor in conditions essentially excluding agitation. The mixture of biologically active compounds constitutes an active ingredient of said composition.

In embodiment, the mixture of biologically active compounds is obtainable from any one of the isolated G. lucidum strains deposited under the Regulations of the Budapest Treaty under accession numbers CBS 147377, CBS 147378, CBS 147379, CBS 147380, or any combination thereof.

In embodiment, the mixture of biologically active compounds is obtainable in liquid phase as a product excreted by mycelium of G. lucidum, wherein the fungi are grown in essentially liquid culture medium in the culture tank or the bioreactor.

In embodiment, the mixture of biologically active compounds obtainable from G. lucidum comprises ganoderic acids and derivatives thereof.

In embodiment, the mixture of biologically active compounds constituting the active ingredient is present in said composition in an amount within a range between about 10 percent to about 99.9 percent of the total volume occupied by the composition.

In embodiment, the composition further comprises hemicellulose. In embodiment, hemicellulose is produced from lignocellulosic biomass. In embodiment, the lignocellulosic biomass is a wood-derived biomass.

In another aspect, a process for producing a virucidal composition comprising a mixture of biologically active compounds obtainable from the selected strains of Ganoderma lucidum is provided, according to what is defined in the independent claim 9.

In embodiment, the process comprises: (a) culturing G. lucidum species in essentially liquid culture medium in a culture tank or a bioreactor; and (b) harvesting the medium as obtained at step (a) and separating the mixture of biologically active compounds as a product excreted by mycelium of G. lucidum in liquid phase optionally followed with concentration or dilution of said product, wherein culturing of the G. lucidum species in essentially liquid culture medium in the culture tank or the bioreactor is implemented in conditions essentially excluding agitation.

In embodiment, the culturing is performed by submerged fermentation.

In embodiment, the culturing of the G. lucidum species in essentially liquid culture medium is performed, at step (a), for a period ranging between 4 weeks to 10 weeks, preferably, for a period of about 6 weeks to 8 weeks.

In embodiment, the process further comprises, at step (c), adding hemicellulose to the product obtained at step (b). In embodiment, hemicellulose is added, at step (c), in an amount within a range of about 0.1 percent to about 75 percent, preferably, within a range of about 5 percent to about 50 percent of the total volume occupied by the composition.

In a further aspect, a preparation for external use in a human or non-human animal subject, comprising the composition according to some previous aspect is provided, according to what is defined in the independent claim 15.

In still further aspect, non-therapeutic use of the composition and/or the preparation according to some previous aspects is provided in preventing the spread of viral infections, according to what is defined in the independent claim 16. In embodiment, said use is provided in preventing the spread of viral infections caused by one or more members of any one of the Picornaviridae and Coronaviridae families.

In further aspects, uses of the composition and/or the preparation according to some previous aspects are provided in disinfecting, sanitizing and/or cleaning, and in manufacturing personal care products or skincare products, according to what is defined in the independent claims 18 and 19, respectively.

In further aspect, a disinfectant for skin or for nonliving surfaces is provided, according to what is defined in the independent claim 20.

In further aspect, a Ganoderma lucidum strain is provided, according to what is defined in the independent claim 21.

In still further aspect, non-therapeutic use of the composition comprising a mixture of biologically active compounds obtainable from the selected Ganoderma lucidum strains in preventing the spread of infections caused by severe acute respiratory syndrome coronavirus 2 (SARS-CoV2) is provided, according to what is defined in the independent claim 22.

The utility of the present invention arises from a variety of reasons depending on each particular embodiment thereof.

At first, the invention provides for a non-toxic composition with pronounced biocidal, in particular, virucidal properties. The composition is particularly suitable for external uses, such as for disinfecting skin and surfaces. The composition can be used as such or as an ingredient in manufacturing a wide variety of personal care items and other end-user products, such as hand sanitizers and general purpose disinfectants, soaps, various moisturizers, cleansing pads, wet wipes and tissues, face masks, textiles for hospitals and for general use, air filters, etc. Obtained in an essentially liquid form, the composition can be used as a cleansing- and/or a disinfecting agent/preparation as such or it can be postprocessed to form a powered preparation or an essentially solid preparation, such as a soap bar, for example. Liquid composition/preparation can be provided in solution or as a spray/aerosol; and it can be further used to in the manufacturing of such items as facial liners or face masks, for example. Semi-solid preparations (skincare/cosmetics products, such as emulsions, creams, etc.) can be further manufactured. The composition is thus versatile and vastly adjustable to the needs of the end user.

A mixture of fungi-derived, biologically active compounds forming an active ingredient of the composition according to the present disclosure has demonstrated marked virucidal activity against a number of virus species including different types of common enteroviruses, coronaviruses, as well as rotaviruses. The present invention thus provides for a broad spectrum antiviral for external use, such as for treating surfaces and objects, as well as for disinfecting hands, for example, to substantially limit the spread of common viruses in public environments, such as hospitals, schools, daycare, malls, airports etc.).

Further, in comparison to bioreactor systems and related processes used in conventional fermentation of fungi, the present invention enables a production process in less energy-intensive manner. This is achieved by eliminating the need in culturing tanks/bioreactors with stirring and/or agitation means. By virtue of implementing the production process in a stationary tank or a bioreactor (wherein stationary refers to the tank or bioreactor not equipped with stirrer(s)/agitator(s)), the method disclosed hereby allows for reducing the equipment costs, as well as costs associated with energy, water and additives consumption, thus improving overall cost-efficiency of the production process and facilitating transition from a pilot scale to a commercial production. Additionally, the present invention allows for avoiding or at least minimizing the measures aiming at optimizing growth morphology of filamentous fungi. Complicated and costly, the growth optimization procedure is mandatory in culturing the fungi in conventional stirred bioreactor systems.

The term “biocidal” commonly refers to a substance that destroys, deters, renders harmless or exerts a controlling effect on any harmful organism.

The term “virucidal” commonly refers to a substance that renders the virus non-viable (neutralizes or destroys the virus). The terms “antiviral” commonly refers to a substance that inhibits replication of otherwise viable virus in the cell and/or prevents viral particles from entering the cell, typically by inhibiting binding to cell receptors or interfering with critical biological processes.

In the context of the present disclosure, the terms “virucidal” and “antiviral” are used interchangeably, wherein the term “antiviral” is largely assigned with a common meaning of “virucidal” to emphasize the ability of a substance to destroy or neutralize viruses, unless explicitly indicated otherwise.

The term “strain” is used hereby to indicate a subtype of fungus characteristically different, in terms of certain genetic elements, for example, from the other fungi within the same species; whereas the term “isolate” is utilized hereby to further emphasize that a number of isolates is obtainable from one fungal strain.

The terms “first” and “second”, are used hereby to merely distinguish an element from another element without indicating any particular order or importance, unless explicitly stated otherwise.

Different embodiments of the present invention will become apparent by consideration of the detailed description.

### BRIEF DESCRIPTION OF THE DRAWINGS

FIG. 1 illustrates the results of screening the initial Ganoderma lucidum samples (2-27) against an infectious agent, hereby coxsackievirus B3 (CVB3), to assess their antiviral activity using cytopathic effect (CPE) inhibition assay.

FIG. 2 illustrates the results confirming antiviral efficacy of selected G. lucidum samples against CVB3 using the CPE inhibition assay.

FIG. 3 illustrates the results of experimental trials aiming at assessing antiviral activity of a selected G. lucidum strain (MUS12) against different dilutions of CVB3 using the CPE inhibition assay.

FIG. 4 illustrates the results of screening the initial G. lucidum samples (2-27) against the infectious agent, hereby coxsackievirus B1 (CVB1), to assess their antiviral activity using the CPE inhibition assay.

FIG. 5 illustrates the results of screening the initial G. lucidum samples (2-27) against the infectious agent, hereby coxsackievirus A9 (CVA9), to assess their antiviral activity using the CPE inhibition assay.

FIG. 6 illustrates the results of experimental trials aiming at assessing antiviral efficacy of selected G. lucidum samples against severe acute respiratory syndrome coronavirus 2 (SARS-CoV2) using a reverse transcription quantitative PCR (RT-qPCR) assay.

FIG. 7 illustrates the results on assessing the effect of treatment parameters (time and temperature) used during (pre)treating the infectious agent with the fungi-derived product on the antiviral activity of said product against the infectious agent (CVA9), using the CPE inhibition assay.

FIG. 8 illustrates the effect of constant agitation on antiviral activity of G. lucidum samples against the infectious agent (CVA9).

FIG. 9A illustrates the results of experimental trials aiming at assessing a potential effect of a hemicellulose compound (HEM) on antiviral activity of selected G. lucidum samples (all MUS18) against the infectious agent (CVA9) using the CPE inhibition assay.

FIG. 9B illustrates the results of experimental trials aiming at determining a dilution threshold, where the selected G. lucidum samples still exhibit antiviral activity against the infectious agent (CVA9) using the CPE inhibition assay.

FIGS. 10A and 10B illustrate the results of screening commercially available Ganoderic Acids A, TQ, Y and TR against the infectious agent (CVA9).

FIG. 11A-F illustrate the results of experimental trials aiming at assessing a potential effect of a hemicellulose compound on antiviral activity of selected G. lucidum samples against the infectious agent (CVA9) using the CPE inhibition assay.

FIGS. 12A and 12B illustrate the results of experimental trials aiming at assessing the effect of incubation period and temperature on antiviral activity of selected G. lucidum samples optionally in presence of a hemicellulose compound against the infectious agent (CVA9) using the CPE inhibition assay. FIG. 12C illustrates the results of experimental trials aiming at assessing cytotoxicity of selected G. lucidum samples optionally in presence of a hemicellulose.

### DETAILED DESCRIPTION OF THE EMBODIMENTS

The present invention pertains to provision of a fungi-derived virucidal composition comprising a mixture of biologically active substances obtainable from selected strains of Ganoderma lucidum fungi, said selected G. lucidum strains, a process for manufacturing the composition, its related uses and end-user products.

The composition comprises, as an active ingredient, a mixture of biologically active compounds, which is further referred to as a fungi-derived product. This product is obtainable by culturing selected G. lucidum strains in essentially liquid culture medium. It is essential that culturing, defining hereby a stage of fungi growth in said liquid media in controlled conditions (a production cycle), is advantageously performed in an absence of agitation and/or stirring. Culturing is advantageously performed using liquid fermentation methods, such as submerged fermentation, for example.

In some embodiment, in addition to the active ingredient, the composition additionally comprises hemicellulose.

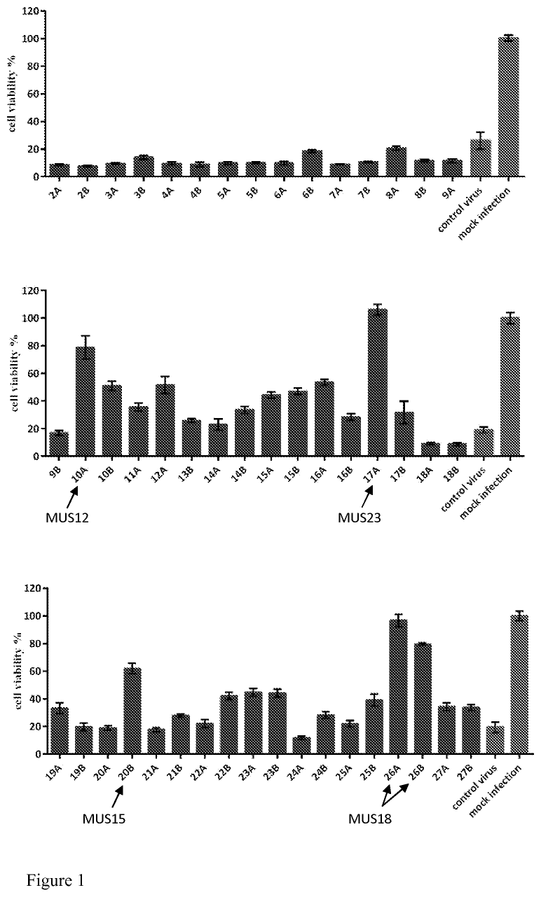

Upon screening through the G. lucidum isolates originating from Finland, the inventors had arrived at a surprising outcome that a limited number of G. lucidum strains were effective in producing, in certain culturing conditions, a metabolite solution possessing strong antipathogenic effect(s) (see screening results of FIGS. 1, 4 and 5). The strains screened for their biological activities were isolated from the fruiting bodies of Ganoderma fungi growing on decayed stumps. During the research studies underlying the present invention it has been found that the product excreted, under certain growth conditions, into the growth/fermentation medium by selected diploid G. lucidum strains exhibited a pronounced antipathogenic activity. In particular, mentioned fungi-derived product extensively inhibited the infectivity of human viral pathogens including, but not limited to the members of the virus families Picornaviridae (enteroviruses) and Coronaviridae (coronaviruses). Additionally, the fungi-derived product appears effective against Reoviridae (rotaviruses), Flaviviridae (virus Zika) and Caliciviridae (noroviruses).

G. lucidum strains that were observed to produce a mixture of biologically active compounds/metabolites, such as ganoderic acids and their derivatives, with pronounced antiviral activity were accorded reference numbers MUS12, MUS15, MUS18 and MUS23. These strains are referred to, in the present disclosure, as selected G. lucidum strains.

The Ganoderma lucidum strains MUS12, MUS15, MUS18 and MUS23 were deposited on 17 Dec. 2020 with the Westerdijk Fungal Biodiversity Institute (CBS, Netherlands) as the International Depository Authority (IDA) under the Regulations of the Budapest Treaty on the International Recognition of the Deposit of Microorganisms for the Purposes of Patent Procedure. The deposit numbers are as follows: CBS 147377 for the strain MUS12; CBS 147378 for the strain MUS15; CBS 147379 for the strain MUS18 and CBS 147380 for the strain MUS23.

Identification of strains MUS12, MUS15, MUS18 and MUS23 has been implemented using DNA-sequencing of multiple gene regions. Phylogenomic analysis (not shown) has revealed that the Ganoderma lucidum fungus collected in Finland is clearly distinct from G. lucidum previously found in China, for example.

The mixture of biologically active compounds is obtained in liquid phase as a product excreted into (liquid) culture medium by mycelium of said selected G. lucidum strains during the production cycle, when the fungi are grown in said medium in a culture tank or a bioreactor. This biologically active product originating from fungi metabolism, includes a group of secondary metabolites, in particular, biosynthesized substances belonging to a triterpenoid class of compounds. At the end of production cycle, the fungi-derived product excreted into the liquid media is separated by filtering, for example, to remove fungal cells and any other insoluble residue, followed with optional concentration or dilution.

Secondary metabolites are typically defined as chemical compounds not indispensable for basic growth and development of a host (on the contrast to primary metabolites) but playing an essential role in survival and competition in the environment.

The biologically active product obtained from the selected G. lucidum strains comprises a group of ganoderic acids (GAs) and their derivatives. Ganoderic acids are a class of closely related triterpenoids, derivatives from lanosterol and widely present in Ganoderma species. Biological activity or activities of said product and the composition comprising the same is/are assumed to be largely underlaid with related activities intrinsic to ganoderic acids and related triterpenoids. However, it has been experimentally demonstrated that only selected strains within G. lucidum species possess pronounced virucidal activity (explained in detail further below).

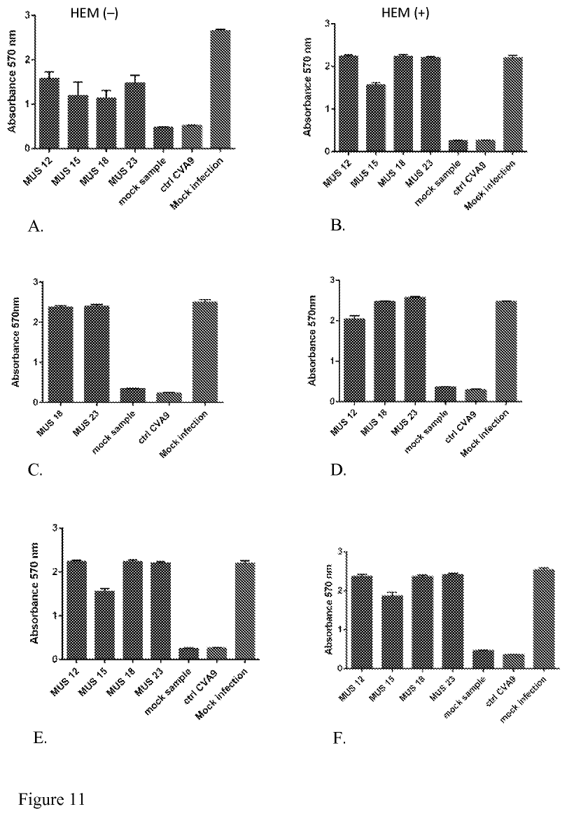

Chemical composition of the fungi-derived product was analyzed by high performance liquid chromatography (HPLC) run with pure ganoderic acid A as a standard (Ganoderic Acid A purchased from ChemFaces, China; Catalog No. CFN92051). Identified compounds simultaneously present in the (secondary) metabolite mixture were different forms of terpenoids, which gave HPLC peaks corresponding to the one assigned to the Ganoderic Acid A standard (not shown). Antiviral effect of the fungi-derived product described herewith was assigned to a combination of these terpenoids and optionally other compounds (e.g. steroids, polysaccharides etc.) excreted into the growth medium. Experimental trials confirmed that only a number of Ganoderma lucidum strains produce antiviral metabolites (FIGS. 1, 4 and 5) and that the pure ganoderic acid used as a standard does not possess any antiviral activity (FIGS. 10A, 10B).

The fungi-derived product obtained from the G. lucidum strains MUS12, MUS15, MUS18 and MUS23 demonstrated virucidal activity against non-enveloped viruses (such as enteroviruses, rotaviruses), as well as against enveloped viruses (such as coronaviruses, virus Zika). Metabolic products obtained from the abovementioned strains were found to possess virucidal effects against individual viral species or combinations of viral species, without showing any toxicity to human cells.

The results of experimental trials demonstrating virucidal activity of the product obtained from the abovementioned G. lucidum strains are described with reference to FIGS. 1-6. Controls included mock infections (negative controls with the cells including neither virus nor G. lucidum derived samples) and control infections (positive controls with the cells infected with virus, but not treated with G. lucidum derived samples). Negative controls are denoted on the figures as “control virus”.

Reference is made to FIGS. 1, 4 and 5 illustrating the results of screening the initial Ganoderma lucidum samples (2-27) against a number of infectious agents to assess antiviral activity of the samples using the cytopathic effect (CPE) inhibition assay. In the trials illustrated by FIGS. 1, 4 and 5, the infectious agents were coxsackieviruses B3, B1 and A9 (CVB3, CVB1 and CVA9; ATCC), respectively. These coxsackieviruses are enteroviruses that belong to the Picornaviridae family (genus Enterovirus). Samples 2-27 were fungi-derived metabolite solutions obtained from different G. lucidum isolates cultured according to the procedure described further below.

FIG. 1 illustrates the results of assessing antiviral activity of selected G. lucidum strains against CVB3. In vitro trials were conducted with a commercially available cell line A549 (human adenocarcinomic alveolar basal epithelial cells), wherein cells at a density of 20,000 cells/well were cultured on 96-well flat bottomed microtiter plates for 24 hours at 37° C. CVB3 (10% v/v) with infectious titre of 7E+06 PFU/ml (plaque forming units/ml) was pre-treated with G. lucidum samples (90% v/v) at 37° C. for 1 hour before cell infection. Final dilution of the virus was 1:50,000 on cells resulting at a MOI (multiplicity of infection) value of 2.3. Infection was allowed to proceed for 24 h at 37° C.

After the incubation, the cells were washed with a neutral buffer (phosphate buffered saline, PBS was utilized) and stained using a CPE dye (CPE dye: 0.03% crystal violet, 2% ethanol, and 36.5% formaldehyde). Stained cells were further washed with water and treated with a lysis buffer (0.8979 g of sodium citrate and 1 N HCl in 47.5% ethanol) solution. Cell viability (%) was determined spectrophotometrically by measuring the absorbance rate at 570 nm on a multilabel plate reader device (Perkin Elmer VICTOR X4). Each of the test and control samples was tested in replicates of three and the values are expressed as mean values+/−Standard Error of the Mean (Mean±SEM). Test samples and positive controls were normalized against mock infections (negative controls).

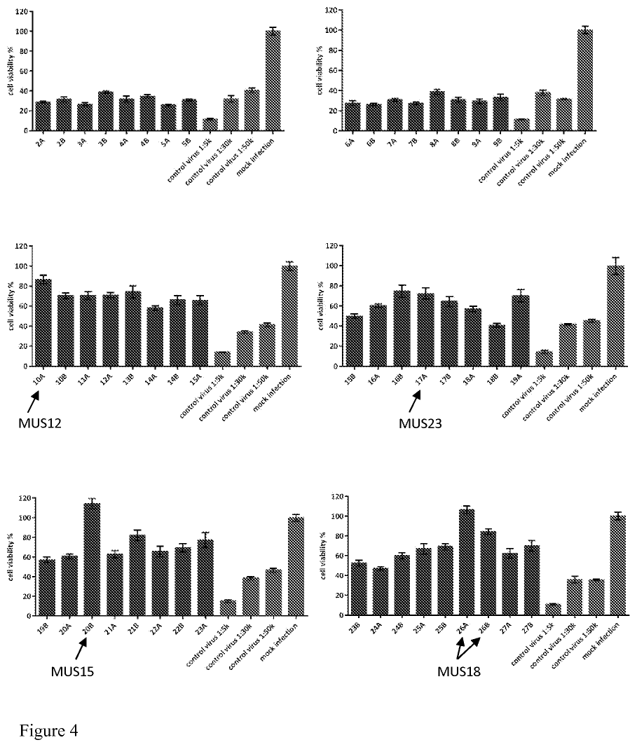

FIG. 4 illustrates the results of assessing antiviral activity of selected G. lucidum strains (2-27) against coxsackievirus B1 (CVB1). Same experimental setup as the one described with reference to FIG. 1 was utilized with an exception that CVB1 infectious titre in a fungi-virus mix was 5E+06 PFU/ml and that the final dilution of virus was 1:30,000 on cells resulting in MOI of 1.6.

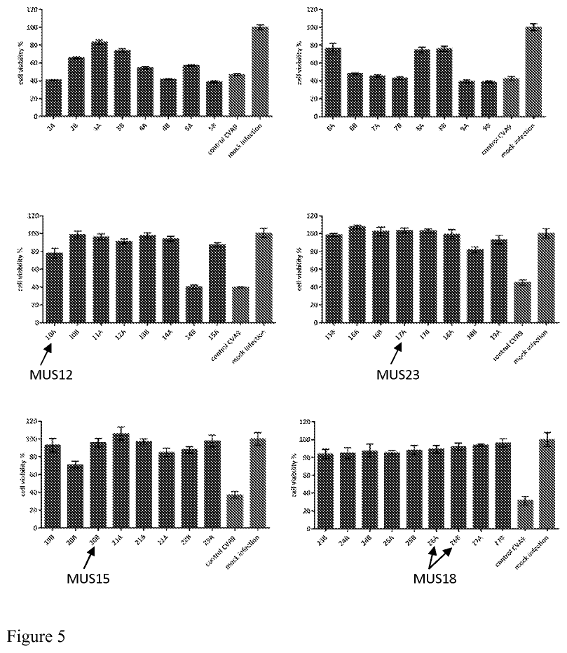

FIG. 5 illustrates the results of assessing antiviral activity of selected G. lucidum strains (2-27) against coxsackievirus A9 (CVA9). Same experimental setup as the one described with reference to FIGS. 1 and 4 was utilized with an exception that CVA9 infectious titre was 3.2E+08 PFU/ml and that the final dilution of 1:5,000 on cells resulted in MOI of 106.

The results presented on FIGS. 1, 4 and 5 clearly indicate that the biologically active products produced by the strains MUS12 (deposit no. CBS 147377; sample identity no. 10A), MUS23 (CBS 147380; sample identity no. 17A), MUS15 (CBS 147378; sample identity 20B) and MUS18 (CBS 147379; sample identity numbers 26A-B) are capable to protect the cells from viral infections induced by dissimilar infectious agents.

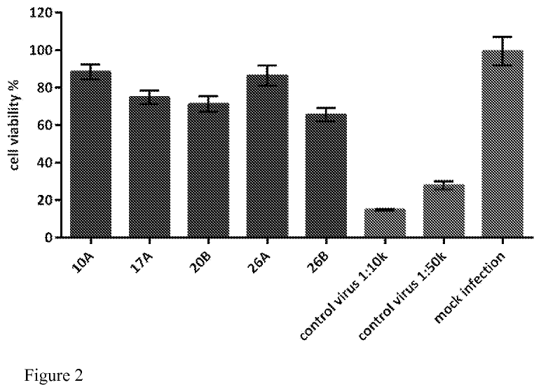

To confirm the antiviral activity of the biological active product derived from the G. lucidum strains MUS12, MUS15, MUS18 and MUS23, these strains were further tested against CVB3 using the same experimental setup as described with regard to FIG. 1. The results are summarized in FIG. 2. The results of FIG. 2 represent the mean value (Mean±SEM) from two (2) independent experiments, where each of the samples (nos. 10A, 17A, 20B, 26A and 26B) was tested in replicates of five. Infectious titre and MOI values were the same as reported for FIG. 1, namely, CVB3 titre in the fungi-virus mix was 7E+06 PFU/ml and final dilution of virus was 1:50,000 on cells resulting in MOI of 2.3. The example of FIG. 2 demonstrates that cell viability in infected cells (cell line A549) treated with the fungi-derived product samples is significantly higher in comparison to that in non-treated infected cells.

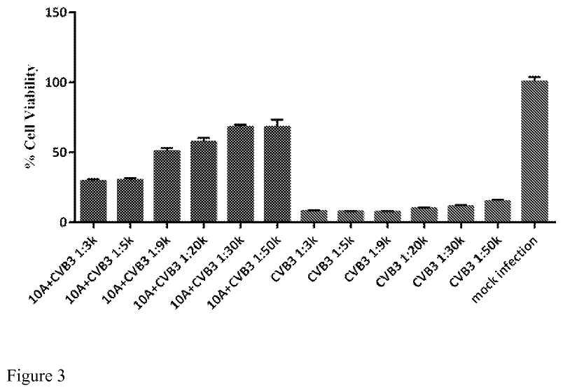

G. lucidum strain MUS12 (deposit no. CBS 147377; sample identity no. 10A) was further selected for assessing its antiviral activity against different dilutions of CVB3 using the same setup as above and the results of these trials are summarized in FIG. 3. The results of FIG. 3 represent the Mean±SEM value from the experiment, where each sample was tested in replicates of five. These results demonstrate that the fungi-derived product is effective in combatting the virus even in dilution of 1:3,000 (“10A+CVB3 1:3k”) resulting in about 30% of viable cells, while the same virus dilution without the fungi-derived product sample kills about 90% of cells (see control sample denoted as “CVB3 1:3k”). About 75% of cells remain viable in infected cell samples, where the virus treated with the fungi-derived product was diluted to the ratio of 1:30,000 and up to 1:50,000 (see “10A+CVB3 1:30k” and “10A+CVB3 1:50k”, respectively). In comparison, infected cell samples served as positive controls have demonstrated only about 15% viability (“CVB3 1:30k” and “CVB3 1:50k”, respectively).

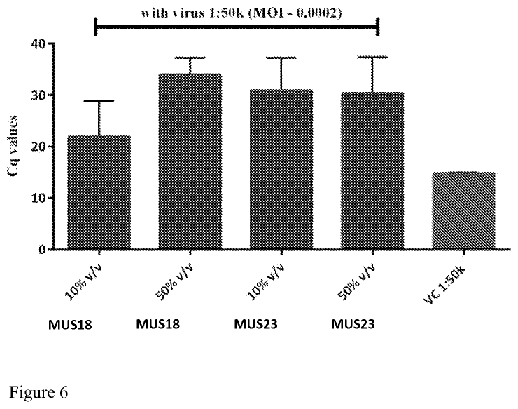

To elucidate whether the antipathogenic effect of the fungi-derived product is preserved across a variety of virus pathogens, similar experiments were conducted against severe acute respiratory syndrome coronavirus 2 (SARS-CoV2) belonging to the Coronaviridae family and defined as a causal agent of coronavirus disease 2019 (Covid-19). The results are summarized in FIG. 6.

The trials referenced at FIG. 6 were conducted with a commercially available Vero cell line (Vero E6). The SARS-CoV2 virus (isolated from a Finnish patient) was pretreated with selected G. lucidum fungi samples obtained from strains MUS18 and MUS23 (1 h, 34° C.). The pretreated virus was subsequently incubated with Vero E6 cells for 3 days; thereafter cell samples were analyzed using a reverse transcription quantitative PCR (RT-qPCR) assay. In brief, viral RNA was extracted from supernatant samples using a commercial RNA extraction kit and detection was made using a Covid19 diagnostic test kit. Both kits were purchased from Perkin Elmer. The quantification cycle (C_{q}) values plotted along a vertical axis (FIG. 6) are an inverse to the amount of target nucleic acid that in the sample; therefore, the lower are the C_{q} values, the higher is the amount of target nucleic acid in the sample and vice versa. From FIG. 6 one may observe that the amount of RNA is high in the control sample (Virus Control, see the sample defined as “VC 1:50k”), while the amount of target RNA has drastically decreased in the test samples (MUS18, MUS23), where virus was pretreated with fungi (higher C_{q} values). The experiment clearly indicated that the selected G. lucidum strains produce a mixture of biologically active compounds effective against the SARS-CoV2 virus.

To confirm that antipathogenic, in particular, antiviral activity of the composition pertains to a mixture of ganoderic acids and their derivatives produced by selected G. lucidum strains cultured in predetermined conditions, as described in the present specification, a number of commercially available ganoderic acids, namely Ganoderic Acids A, TQ, Y and TR, purchased from ChemFaces, China (Catalog No. CFN92051 for GA A, Catalog No. CFN92237 for GA TQ, Catalog No. CFN90294 for GA Y, and Catalog No. CFN92235 for GA TR) has been tested against the infectious agent. The results are summarized in FIGS. 10A and 10B, where FIG. 10A illustrates the experiments with Ganoderic Acid A and FIG. 10B illustrates the experiments with Ganoderic Acids TQ, Y and TR.

The commercial GAs were screened against CVA9 to determine their antiviral activities in different concentrations (100 μM, 500 μM, 1 mM, 5 mM and 10 mM for GAA; 12.5 μM, 25 μM, 50 μM, 100 μM, and 500 μM for GAs TQ, Y and TR;). Infectious titres with respect to the mixtures of the virus with GAs TQ, Y, TR and the mixture of the virus with GA A were 2E+08 PFU/ml and 1.6E+08 PFU/ml, respectively, resulting in the MOI values 100 and 66.7, respectively. CVA9 was treated with the GA samples at 37° C. for 1 hour before infecting cells (cell line A549). The results show that the commercial ganoderic acid product fails to demonstrate any antiviral activity against the infectious agent.

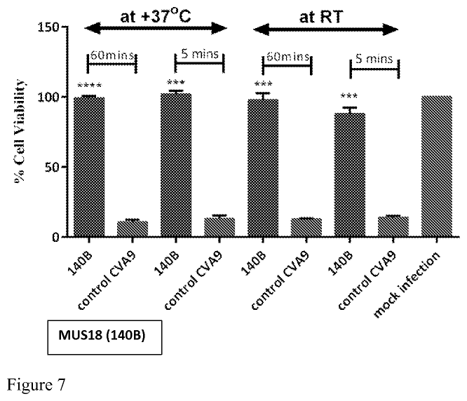

Reference is made to FIG. 7, showing the results on assessing the effect of treatment parameters (treatment duration and temperature) used during (pre)treating the infectious agent with the fungi-derive product on the virucidal activity of the latter against the infectious agent. In vitro trials were conducted with the cell line A549, wherein cells at a density of 12,000 cells/well were cultured on 96-well flat-bottomed microtiter plates for 24 hours at 37° C. The infectious agent, hereby CVA9 (10% v/v), with virus titre of 3.2E+08 PFU/ml was (pre)treated with G. lucidum derived samples (90% v/v). In the experiment, the fungi-derived product was produced from the strain MUS18 (CBS 147379; sample identity number 140B). Treatments were conducted in different time intervals (5 min and 60 min) and at different temperatures, namely, at 37° C. and at room temperature (RT, 23-24° C.). The virus-sample mixture was further diluted by a factor of ten (10) to reach the MOI value of 130 and the cells were infected with the fungi-treated virus, followed with an incubation period of 24 h at 37° C.

After the incubation, the cells were washed with PBS and stained using CPE dye. Stained cells were washed with water and treated with a lysis buffer solution. Cell viability (%) was determined spectrophotometrically by measuring absorbance rate at 570 nm on a multilabel plate reader device (Perkin Elmer VICTOR X4). The results are Mean±SEM values from two independent experiments. Statistical significance of differences between the test samples and the positive control was assessed using one-way ANOVA test followed by Bonferroni test. Based on the results, it is evident that the fungi-derived product obtained from the selected G. lucidum strains (herein, G. lucidum derived samples) demonstrates pronounced virucidal activity against the infectious agent even when the treatment of said infectious agent with the fungi-derived extract is conducted during a short time interval (5 min). Virucidal activity of the selected G. lucidum strains remained essentially unchanged no matter whether the infectious agent was treated with the fungi-derived product at room temperature (23-24° C.) or at 37° C.

The following describes a process for producing the composition comprising the fungi-derived product obtainable from the selected Ganoderma lucidum strains.

The process involves culturing the selected G. lucidum strains in in essentially liquid culture medium in a culture tank or a bioreactor. The fungi cultures are allowed to grow in the culture tank during a predetermined time period and in certain conditions, involving incubation temperature, growth media related parameters, presence/absence of (day)light etc. Fungi growth during said predetermined period of time in certain conditions is referred to as the production cycle. The production process is fully scalable within a range of e.g. 2 L (2 liter) tanks up to industrial-scale reactors having a volume of 10-100 L or higher. Volumes under 2 L are not excluded, but those are typically not feasible for industrial cultivation. Production cycle may be preceded with pre-culturing the fungi strains in smaller volumes using same liquid culture medium. By way of example, pre-cultures grown in 0.1 L Erlenmeyer flasks were inoculated into 2 L tanks, according to a well-established procedure.

Exemplary growth (fermentation) medium contains D-glucose (35 g), peptone (5 g), yeast extract (2.5 g), salts and vitamins, such as monopotassium phosphate KH₂PO₄ (1 g), magnesium sulfate heptahydrate MgSO₄×7H₂O (0.5 g), and thiamine (0.05 g), accordingly. The above amounts are given for 1000 ml of water. Solution pH is 5.5. Use of other appropriate growth media is not excluded.

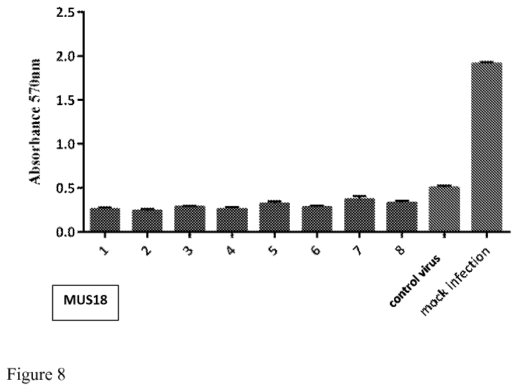

For producing the composition with pronounced antiviral activity, it is further essential that culturing of the fungi strains during the production cycle (at step a) is implemented in conditions essentially excluding agitation (see description to FIG. 8). It has been shown that fungi strains allowed to grow in stationary conditions for a period between 4 weeks to 10 weeks, preferably, for a period of about 6 weeks to about 8 weeks, demonstrate optimum antipathogenic behavior. Longer culturing periods (up to 6 months) are possible in terms of producing a desired product; however, such long periods may not be feasible in industrial implementation. Optimum growth conditions included temperature within a range of 20-25° C. and an absence of (day)light. The exemplary 8 weeks production cycle was preceded with a one week pre-culturing period. It is assumed that the skilled person would not experience any difficulties in adjusting the duration of pre-culturing period vs production cycle within the above identified limits.

During the production cycle, the fungi are advantageously cultured by liquid fermentation methods, such as submerged fermentation, for example. Any other appropriate method is not excluded.

With reference back to the production method, it has been established that the selected G. lucidum strains (MUS12, MUS15, MUS18 and MUS23) cultivated in essentially liquid culture medium in the culture tank or bioreactor produce biologically active products with pronounced antipathogenic properties only in conditions that essentially exclude agitation.

In conventional systems, filamentous fungi, like Ganoderma species, are grown in stirred bioreactors. However, the research underlying the present invention has led the inventors to an unexpected outcome that in conventional production setup involving specialized bioreactors/fermenters equipped with stirrers or other agitation means, the excretion of the antiviral compounds is inhibited.

In particular, it has been demonstrated that conditions of constant stirring throughout the entire production cycle optionally conducted in a conventional bioreactor setup (e.g. a bioreactor, such as a conventional fermenter, equipped with a stirrer) is/are not suitable for the production of antiviral compounds, according to the concept of the present invention. The fungi-derived compounds produced in such conventional stirred fermenters have failed to demonstrate antiviral activity, thus indicating that conditions involving agitation and/or stirring are not suitable for the production of the antiviral composition.

The effect of constant agitation on antiviral activity of the biologically active product obtained from G. lucidum (strain MUS18) against the infectious agent is demonstrated by FIG. 8. A process of fungal growth/-fermentation was conducted following the guidelines outlined above, but in stirred reactors (the reactors equipped with stirring means). In brief, the trials were conducted in 2 L batch fermenters with a two-month (about 8 weeks) production cycle. The latter was preceded with 7 days of preculturing the strains in 0.1 L Erlenmeyer flasks (25° C., 120 rpm, in a dark room). During the growth/production cycle MUS18 samples were harvested at regular time intervals (once a week for 8 weeks) and tested against coxsackievirus A9 (virus titre 3.2E+08 PFU/ml; MOI 133) to evaluate their antiviral activity using CPE inhibition assay. Conditions in batch fermenters were as follows: room temperature+25° C. in an absence of (day)light and constant stirring (120 rpm). Same growth medium was used as for the stationary fermentation. The pH and oxygen (O₂) levels were monitored (not adjusted) during cultivation; however, no significant changes were observed.

The samples collected in conditions of constant agitation throughout the growth cycle have failed to demonstrate the antiviral activity, on the contrary to the samples collected during culturing excluding agitation. Hence, it has been experimentally proven that when selected G. lucidum strains are cultured/fermented in stirred bioreactors, in conditions of agitation, the formed mixture of compounds (metabolites) does not possess antipathogenic, hereby antiviral, activity.

Agitation of growth media during pre-culturing has not influenced the antipathogenic activity of fungi-derived metabolites. In described setup, 0.1 L flasks were allowed to incubate on an orbital shaker at moderate speed (120 rpm).

It has been observed that during the growth period defining the production cycle herewith, i.e. the period of about 4-10 weeks, preferably, about 6-8 weeks the cultured fungi strains release into the liquid growth medium an optimum amount of biological compounds/metabolites of interest. After the production cycle the medium is harvested by removal the medium from the culture tank or the batch reactor, for example, and a crude product excreted by mycelium of G. lucidum into liquid phase is separated from fungi cells and insoluble debris by conventional techniques, such as centrifugation and/or (sterile) filtering. Harvesting and separation stage subsequent to the production cycle is referred to as a production process step b. During separation, the crude metabolite product is purified to yield the active ingredient for the composition according to the embodiments. Separated/purified product can be optionally subjected to further concentration or dilution. Concentration may be performed by freeze-drying, for example. Dilution can be performed with any appropriate dilution medium. Exemplary dilution media include phosphate- and chloride based buffer solutions, such as PBS optionally containing magnesium chloride (MgCl₂). In some particular examples, PBS containing 2 mM magnesium chloride has been utilized.

In embodiments, the fungi-derived composition consists of a mixture of biologically active compounds/metabolites obtained at step b of the above described process and referred to as the fungi-derived biologically active product. In some other embodiments, the composition further comprises at least one hemicellulose compound or a mixture of different hemicellulose compounds.

The effect exerted on viral particles by said biologically active fungal metabolites is manifested through stabilizing the virus particles and strongly clustering them. By addition of hemicellulose to the biologically active product biosynthesized in metabolic pathways of the selected G. lucidum fungi strains, additional biological capacities are imparted to the yielded composition, such as antioxidant capacity and enhanced mechanical stability. Typically, hemicellulose acts as a carrier/stabilizer.

Hemicellulose compound(s) is/are admixed to the separated and optionally concentrated or diluted fungi-derive biologically active product obtained at step b of the process disclosed hereinabove. Admixing of the hemicellulose(s) can be realized directly after separation/purification (and optional concentration or dilution) of the fungi-derived product to yield the composition. The fungi-derived product becomes diluted to some extent when hemicellulose is added thereto, in particular, when the added hemicellulose is provided in solution.

Alternatively, hemicellulose can be admixed into the fungi-derived product shortly prior use. A kit is therefore provided, comprising the fungi-derived biologically active product as a first component and at least one hemicellulose compound as a second component, said first- and second components to be combined with one another at any timepoint prior to their collective use. In the kit, the ratio between the fungi-derived active ingredient and the hemicellulose compound is maintained essentially the same as disclosed for the composition.

The at least one hemicellulose compound is advantageously produced from lignocellulosic biomass. Hemicellulose compound can be produced from any one of wood- or non-wood biomass; still in some instances, wood-derived biomass is preferred. For the purposes of the present invention, the lignocellulosic biomass can be produced from deciduous trees/hardwood (e.g. birch, Betula ssp.), coniferous trees/softwood (spruce, Picea ssp.; pine, Pinus ssp.) and/or the mixtures of those. In exemplary production trials, the hemicellulose compound was produced from the mixtures of birch and spruce, birch and pine, spruce and pine and a mixture of birch, spruce and pine.

In some instances, the hemicellulose compound(s) comprise mannans, xylans and any combination thereof. Mannan polysaccharides are represented primarily by galactoglucomannans (GGMs) and glucomannans (GMs), while xylans are represented primarily by glucuronoxylan (GX) and glucuronoarabinoxylan (GAX). We note that while xylans typically occur in hardwoods, the primary hemicellulose polysaccharides in softwoods are mentioned glucomannan or galactoglucomannan.

The hemicellulose compound(s) can be obtained with suitable extraction methods, such as steam extraction, hot-water extraction or with any other appropriate method.

In some instances, the composition further comprises pectin, such as galatouronic acid and/or rhamnose.

A portion of the fungi-derived biologically active product in the composition is within a range of about 10 percent to about 99.9 percent, in some embodiments, within a range of about 50 percent to about 75 percent, of the total volume occupied by the composition, taken that the composition is provided in essentially liquid phase, such as solution or suspension. In some instances, the portion of the fungi-derived active product in the composition is at least 25 percent of the total volume occupied by the composition.

A portion of the hemicellulose compound is said composition is within a range of about 0.1 percent to about 75 percent, preferably, within a range of about 5 percent to about 50 percent, of the total volume occupied by the composition.

The hemicellulose component thus acts as a dilution medium for the fungi-derived biologically active product in the composition. In addition to or instead of hemicellulose, the composition comprises some other dilution media, such as phosphate- and/or chloride based buffer or buffers (PBS, MgCl₂), water or a clean growth medium solution.

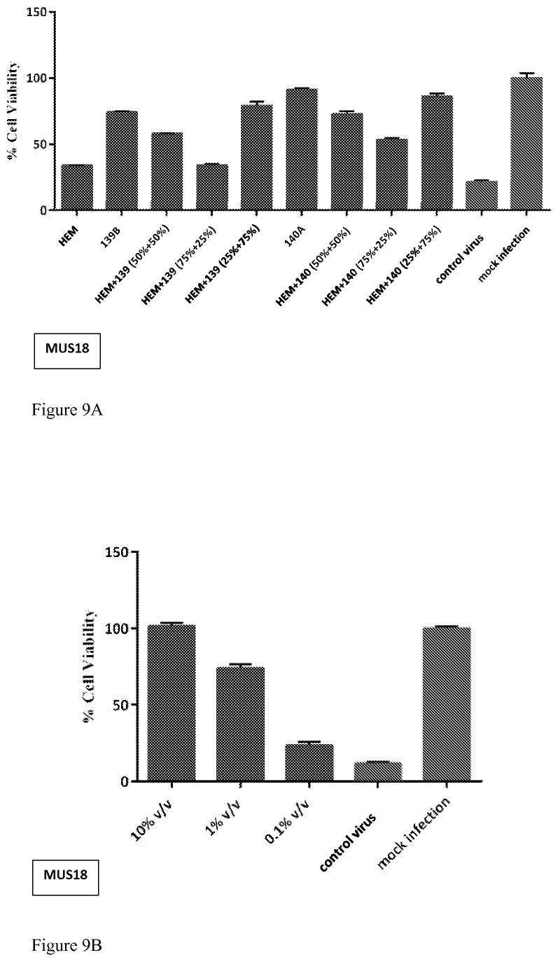

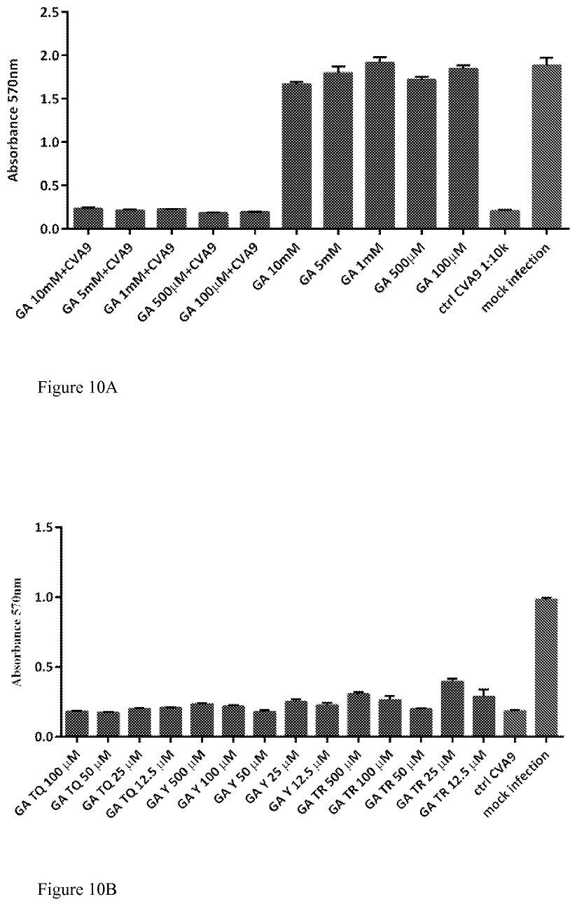

Hemicelluloses have excellent properties as emulsions. They enhance structural properties of the composition (such as consistency, for example). Additionally, hemicelluloses have strong antioxidant properties and long shelf-life. The experimental trials have shown that hemicelluloses do not interfere with the biological (hereby, antiviral) activity of the fungi-derived biologically active product. In fact, hemicelluloses support and stabilize said antiviral activity. The composition, where the fungal-derived biologically active product is diluted with hemicellulose by half (in a ratio of 50:50) does not lose its antipathogenic/antiviral activity (FIG. 9A).

Reference is made to FIG. 9A illustrating the results of experimental trials aiming at assessing a potential effect of the hemicellulose compound (HEM) on antiviral activity of selected G. lucidum samples (all fungi samples referred to in the FIG. 9A are MUS18, CBS 147379) against the infectious agent (CVA9) using CPE inhibition assay. In the trials, the hemicellulose compound was birch-derived hemicellulose and the infectious agent was CVA9 (infectious titre 1.6E+09 PFU/ml). The test samples and the controls were tested in replicates of three and the values are expressed as Mean±SEM. The results indicate that the composition comprising the G. lucidum derived product admixed with hemicellulose in a percent ratio of 75:25 up to 50:50, respectively, possesses good antiviral activity and is capable of protecting the cells against the infectious agent(s).

FIG. 9B illustrates the results of experimental trials aiming at determining a dilution threshold, where the selected G. lucidum derived samples still exhibit antipathogenic activity. Dilution series of selected G. lucidum samples (strain MUS18) with the PBS buffer containing 2 mM MgCl₂ have been prepared and tested against the infectious agent (hereby, CVA9) using cytopathic effect (CPE) inhibition assay. Same experimental setup as described hereinabove was utilized with the exception that the infectious agent (CVA9 virus titre 2×108 PFU/ml and the MOI 100) was pre-treated with a number of fungi-derived samples diluted in series (10% v/v, 1% v/v, 0.1% v/v). Cell line A549 was utilized. Each of the test and control samples was tested in replicates of three and the values are expressed as Mean±SEM. The results show that the composition comprising about 10 percent by volume of the fungi-derived substance (the rest being the dilution buffer) demonstrates 100% cell viability. Even in lower amounts (e.g. 1% v/v) the fungi-derived active product reliably demonstrates its antiviral activity (resulting in about 75% of viable cells) and its ability to protect cells from virus infection.

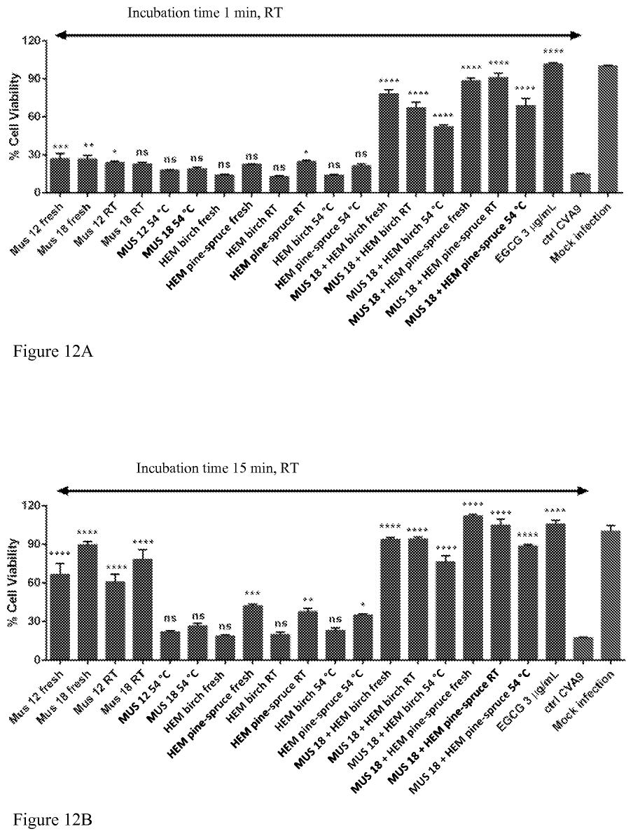

FIGS. 11A-11F illustrate the results of experimental trials aiming at assessing a potential effect of the hemicellulose compound added into the liquid culture medium in a culture tank or bioreactor on antiviral activity of G. lucidum samples (MUS12, MUS15, MUS18 and MUS23) against the infectious agent (CVA9) using the CPE inhibition assay. In all experiments, the hemicellulose compound was added in a concentration of 1.5% (v/v). The following controls were utilized: a positive control (denoted as “ctrl CVA9”) which included cells infected with a virus but not treated with tested samples; a negative control (denoted as “mock infection”) which included cells without the virus and without the tested samples; and an additional control (denoted as “mock sample”) which included only the growth medium tested with the virus.

FIGS. 11A and 11B show comparative results for assessing the antiviral activity of the G. lucidum samples against CVA9 with and without hemicellulose. Fungi strains were thus grown in a standard media (with no hemicellulose added; FIG. 11A) and in a standard media with the added hemicellulose compound (1.5% v/v; hardwood hemicellulose, namely birch xylan, was utilized; FIG. 11B). Concentration of the fungi-derived product in a mixture including the infectious agent and the hemicellulose compound (virus-compound mix) was 80% v/v. CVA9 virus titre was 2×106 PFU/ml and the MOI was 1. Samples were incubated for 1 hour at 37° C. The result is expressed as Mean±SEM. The result demonstrates that the hemicellulose compound supports the antiviral activity of the fungi strains.

FIGS. 11C-11F involve the use of hemicellulose together with the fungi-derived product.

FIG. 11C illustrates the results of experimental trials aiming at assessing the antiviral activity of the G. lucidum strains MUS18 and MUS23 against CVA9. Fungi strains were grown in the standard media supplemented with the hemicellulose compound (1.5% v/v; birch xylan). Concentration of the fungi-derived product in a mixture including the infectious agent and the hemicellulose compound was 80% v/v. CVA9 virus titre was 2×106 PFU/ml and the MOI was 1. Samples were incubated for 1 hour at 37° C. Each of the test and control samples was tested in replicates of five and the values are expressed as Mean±SEM.

FIG. 11D illustrates the results of experimental trials aiming at assessing the antiviral activity of the G. lucidum strains MUS12, MUS18 and MUS23 against CVA9. In this experiment, the hemicellulose compound was softwood hemicellulose (a mixture of hemicellulose compounds obtained from pine and spruce), added into the growth medium in an amount of 1.5% v/v Concentration of the fungi-derived product in a mixture including the infectious agent and the hemicellulose compound was 80% v/v. CVA9 virus titre was 2×106 PFU/ml and the MOI was 1. Samples were incubated for 1 hour at 37° C. The result is expressed as Mean±SEM.

FIG. 11E illustrates the results of experimental trials aiming at assessing the antiviral activity of the G. lucidum strains MUS12, MUS15, MUS18 and MUS23 against CVA9. The hemicellulose compound was hardwood hemicellulose (birch xylan), added into the growth medium in an amount of 1.5% v/v. Concentration of the fungi-derived product in a mixture including the infectious agent and the hemicellulose compound (virus-compound mix) was 80% v/v. CVA9 virus titre was 2×106 PFU/ml and the MOI was 1. Samples were incubated for 1 hour at 37° C. The result is expressed as Mean±SEM.

FIG. 11F illustrates the results of experimental trials aiming at assessing the antiviral activity of the G. lucidum strains MUS12, MUS15, MUS18 and MUS23 against CVA9. The hemicellulose compound was softwood hemicellulose (a mixture of hemicellulose compounds obtained from pine and spruce), added into the growth medium in an amount of 1.5% v/v. Concentration of the fungi-derived product in a mixture including the infectious agent and the hemicellulose compound (virus-compound mix) was 80% v/v. CVA9 virus titre was 2×108 PFU/ml and the MOI was 100. Samples were incubated for 1 hour at 37° C. The result is expressed as Mean±SEM.

The results of FIGS. 11C-11F indicate that hemicellulose compounds obtained from both hardwood- (birch, FIGS. 11C, 11E) and softwood (pine-spruce, FIGS. 11D, 11F) added to the production process support the antiviral activity of fungi-derived products and ability thereof to protect cells from viral infection.

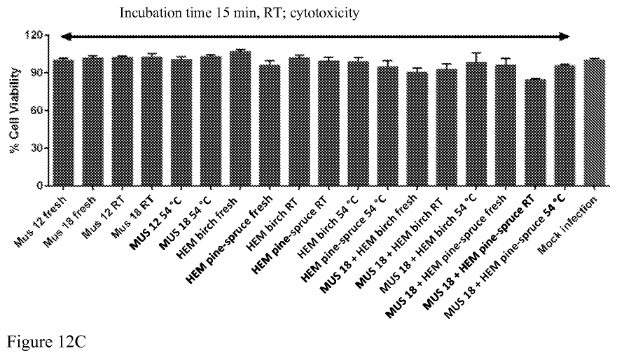

That the hemicellulose compound has a stabilizing effect on antiviral properties of the fungi-derived product is further demonstrated by the results shown on FIGS. 12A and 11B.

FIGS. 12A and 12B illustrate the results of experimental trials aiming at assessing the effect of incubation period and temperature on antiviral activity of selected G. lucidum samples optionally in presence of a hemicellulose compound against the enterovirus coxackievirus A9 (CVA9) using the CPE inhibition assay. Stability of the fungi-derived product when admixed with hemicellulose was assessed.

In the trials, G. lucidum strains MUS12 and MUS18 were tested alone and with the hemicellulose compounds obtained from hardwood and softwood (generally indicated as “HEM birch” and “HEM pine-spruce”, respectively). Incubation conditions were selected to preserve the antiviral effect expressed by the fungi-derived product at a relatively low level in order to detect any additional effects potentially originating from hemicellulose. These conditions included, for example, high infectious titre of 2×106 PFU/ml and optionally short incubation time (1 min, rf. FIG. 12A).

The fungi-derived product samples, hemicellulose samples and mixtures thereof were pre-incubated at different temperature regimes (room temperature and 54° C.) for two weeks. The infectious agent (CVA9) was admixed to said samples according to the following recipe: 10% of the fungi-derived product, 25% of the hemicellulose compound, and CVA9 in a buffer solution (PBS-MgCl₂) in a total volume of 100 microliter.

The cells (cell line A549, ATCC) were cultured on 96-well plates (12,000 cells/well) for 24 hours at 37° C. On day 2, a mixture of the infectious agent (CVA9) and the compounds (fungi-derived products optionally supplemented with hemicellulose), as described above, was prepared in a suitable buffer (e.g. PBS—MgCl₂) and incubated at a room temperature (RT) for 1 min and for 15 min. This mixture was diluted by a factor of 10 using 10% DMEM (Dulbecco's Modified Eagle Medium) and added to the cells to achieve the final MOI value of 1. The cytopathic effect was allowed to develop during two days at 37° C. Hence, after 48 hours, the cells were washed twice with PBS, followed with fixing and staining for 10 min using CPE dye (0.03% crystal violet, 2% ethanol and 36.5% formaldehyde). After staining and washing the wells with water, cells were lysed using a lysis buffer (0.8979 g of sodium citrate and 1N HCl in 47.5% ethanol) to elute the crystal violet. The absorbance of viable cells was measured spectrophotometrically at 570 nm using the PerkinElmer VICTOR™ X4 multilabel reader.

Epigallocatechin gallate (EGCG) in an amount of 3 μg/mL was used as an additional positive control.

The results of experiments, where the cells were treated with the virus-compound mixture during 1 min at RT are shown on FIG. 12A.

After a short incubation period (1 min), the samples containing the fungi-derived product (10%) alone, as well as the samples containing hemicelluloses derived from hardwood (HEM birch) and softwood (HEM pine-spruce) were not able to effectively protect against the CVA9 infection with high infectious titre. In addition, the results from the samples containing the fungi-derived product alone and the hemicellulose compound alone showed no difference. In contrast, the hemicellulose compound added to the fungi-derived product has demonstrated a high level of protection against the viral infection (see a group of samples generally designated with “MUS18+HEM”).

The fungi-derived products and the hemicellulose compounds pre-incubated at 54° C. for two weeks do not possess protective properties against the viral infection. However, a mixture of the fungi-derived product and the hemicellulose compound pre-incubated for two weeks at 54° C. has demonstrated good antiviral protection.

The results obtained in a 1 min treatment show no statistically significant difference between different hemicellulose species when freshly taken from the stock. However, the softwood hemicellulose species (pine-spruce) demonstrated statistically better results than hardwood hemicellulose species (birch) at RT (**=p<0.01) and at 54° C. (*=p<0.05). As expected, the positive controls have demonstrated high viability of cultured cells, whereas the control infection (ctrl CVA9) have demonstrated low viability of cells. Designations “ns” stand for “non-specified”.

The results of FIG. 12B demonstrate that the stabilizing effect exerted by hemicelluloses onto antiviral efficacy of the fungi-derived product is retained also during a longer (15 min) incubation period with the virus.

Experimental conditions were generally the same as described with regard to FIG. 12A. The results of FIG. 12B demonstrate that fungi-derived products alone were already effective at RT. However, the fungi-derived products alone and the hemicellulose species alone pre-treated at 54° C. (for two weeks) had low antiviral efficacy (see samples MUS 12 54° C., MUS 18 54° C., HEM birch 54° C. and HEM pine-spruce 54° C.). In contrast, adding hemicellulose to the fungi-derived products demonstrated excellent protection against the viral infection even after the 54° C. treatment. In present experiment, the hemicellulose compound derived from softwood (pine-spruce) was statistically better than that derived from hardwood (birch) at 54° C. (*=p<0.05) and when used fresh (**=p<0.01), but demonstrated no significant difference at RT.

FIG. 12C illustrates the results of experimental trials aiming at assessing cytotoxicity of selected G. lucidum samples optionally in presence of hemicellulose. These results indicate that the fungi-derived products and hemicellulose show no cytotoxicity against cells.

The products derived from G. lucidum strains MUS12 and MUS18 and the hemicellulose compounds derived from hardwood (birch) and softwood (pine-spruce), and combinations thereof were tested directly on cells to assess their potential cytotoxicity after treatments conducted at different temperatures. The results of FIG. 12C demonstrate that none of the samples was cytotoxic.

The results of FIGS. 9A, 11A-F and 12A-B clearly demonstrate that the hemicellulose compounds support antiviral activity of biologically active products derived from selected G. lucidum strains and exert stabilizing effect on the fungi-derived products thus allowing the resulted composition to be stable in liquid form which is particularly suitable for external use.

In another aspect, a preparation for external use in a human or non-human animal subject is provided, said preparation comprising or consisting of the composition, which comprises the mixture of biologically active compounds obtainable from the selected strains of Ganoderma lucidum fungi as per the embodiments described hereinabove.

In addition to the components that make up the composition, the preparation can further comprise at least one of a solvent, an additive, a preservation agent, a thickener, or a combination thereof. The preparation may be provided in an essentially liquid form (solutions, suspensions), in semi-solid form (emulsions, creams) or in solid form (e.g. a soap bar). Moreover, the preparation may be provided as a powder, spray or aerosol. Powdered preparation can be further pressed to form an item used in decorative cosmetics.

In some preferred configurations, the preparation is provided as a cosmetics product, such as a skincare product (creams, serums etc.) or as a decorative cosmetics item. Mentioned cosmetics product(s) is/are preferably imparted with at least one additional functionality, such as anti-age functionality, for example.

In various configurations, the preparation is provided as a disinfectant for skin or for nonliving surfaces, a cleaning agent, a sanitizer, and the like.

In an aspect, the invention pertains to a use of any one of said composition and preparation in disinfecting, sanitizing and/or cleaning. Thus, use of the composition and/or the preparation in treating (non-living) surfaces and objects, as well as for skin and hair disinfection in human and non-human animal subjects is provided. In some configurations, use of said composition and/or the preparation is provided in air cleaning applications, in particular, for treating air filters, such as the ones used in air exchangers and/or in air conditioning systems.

In another aspect, the invention pertains to a use of any one of said composition and preparation in manufacturing personal care products and/or skincare products. The personal care product(s) include, but are not limited to cleansing pads, cotton pads, wet wipes and tissues, facial liners, face masks, infant care products, textiles, such as hospital textiles and/or personal use textiles, air filters for personal use, and the like.

In further aspect, the invention pertains to non-therapeutic use of the composition and/or the preparation as per described embodiments in preventing the spread of viral infections.

In particular, the composition and the related preparation are beneficial for preventing the spread of viral infections caused by one or more members of any one of the Picornaviridae, Coronaviridae, Reoviridae and Flaviviridae. As described with reference to FIGS. 1-6 and 9A, 9B the composition comprising fungi-derived extract and optionally the hemicellulose compound(s) demonstrates pronounced virucidal activity with respect to different types of coxsackievirus and SARS-CoV2 related to coronaviruses.

Mechanism of function of the fungi-derived triterpenoid-containing products on picornaviruses (including enteroviruses), and coronaviruses is presumably based on a direct action of said products on the virions. Some of triterpenoid-based compounds delivered by the selected G. lucidum strains disclosed hereby are assumed, according to some literature sources, to act on characteristic sites or conserved amino acid regions common to viral proteases and through that exert an inhibitory action. The fungi-derived product, according to the present disclosure, appears to exert its virucidal activity also on rotaviruses and flaviviruses based on structural similarities between enveloped and non-enveloped viruses.

In further aspect, a Ganoderma lucidum strain is provided, said strain selected from the group consisting of the strains deposited under the Regulations of the Budapest Treaty under accession numbers CBS 147377, CBS 147378, CBS 147379 and CBS 147380.

In still further aspect, non-therapeutic use of a composition comprising a mixture of biologically active compounds obtainable from any one of the isolated Ganoderma lucidum strains deposited under the Regulations of the Budapest Treaty under accession numbers CBS 147377, CBS 147378, CBS 147379, CBS 147380, or any combination thereof is provided in preventing the spread of infections caused by severe acute respiratory syndrome coronavirus 2 (SARS-CoV2).

It is clear to a person skilled in the art that with the advancement of technology the basic ideas of the present invention may be implemented and combined in various ways. The invention and its embodiments are thus not limited to the examples described herein above, instead they may generally vary within the scope of the claims.

1. A virucidal composition comprising a mixture of biologically active compounds obtainable from selected strains of Ganoderma lucidum by culturing said fungi in essentially liquid culture medium in a culture tank or a bioreactor in conditions essentially excluding agitation.

2. The composition of claim 1, wherein the mixture of biologically active compounds is obtainable from any one of the isolated G. lucidum strains deposited under accession numbers CBS 147377, CBS 147378, CBS 147379, CBS 147380, or any combination thereof.

3. The composition of claim 1, wherein the mixture of biologically active compounds is obtainable in liquid phase as a product excreted by mycelium of G. lucidum, wherein the fungi are grown in essentially liquid culture medium in the culture tank or the bioreactor.

4. The composition of claim 1, wherein the mixture of biologically active compounds obtainable from G. lucidum comprises ganoderic acids and derivatives thereof.

5. The composition of claim 1, wherein the mixture of biologically active compounds is present in an amount within a range between about 10 percent to about 99.9 percent of the total volume occupied by the composition.

6. The composition of claim 1, further comprising hemicellulose.

7. The composition of claim 6, wherein hemicellulose is produced from lignocellulosic biomass.

8. The composition of claim 7, wherein the lignocellulosic biomass is a wood-derived biomass.

9. A process for producing a virucidal composition comprising a mixture of biologically active compounds obtainable from selected strains of Ganoderma lucidum, the process comprises:
a. culturing G. lucidum species in essentially liquid culture medium in a culture tank or a bioreactor; and
b. harvesting the medium as obtained at step (a) and separating the mixture of biologically active compounds as a product excreted by mycelium of G. lucidum in liquid phase,
wherein culturing of the G. lucidum species in essentially liquid culture medium in the culture tank or the bioreactor is implemented in conditions essentially excluding agitation.

10. The process of claim 9, wherein the selected strains of Ganoderma lucidum are any one of the isolated G. lucidum strains deposited under accession numbers CBS 147377, CBS 147378, CBS 147379, CBS 147380, or any combination thereof.

11. The process of claim 9, wherein the culturing is performed by submerged fermentation.

12. The process of claim 9, wherein culturing of the G. lucidum species in essentially liquid culture medium is performed for a period ranging between 4 weeks to 10 weeks, weeks.

13. The process of claim 9, further comprising, at step (c), adding hemicellulose to the product obtained at step (b).

14. The process of claim 13, wherein hemicellulose is added in an amount within a range of about 0.1 percent to about 75 percent of the total volume occupied by the composition.

15. A preparation suitable for external use in a human or non-human animal subject, comprising the composition as defined in claim 1.

16. A method for preventing the spread of viral infections, comprising applying an effective amount of the composition of claim 1 to a patient in need thereof.

17. The method of claim 16, wherein said viral infection is caused by one or more members of any one of the Picornaviridae and Coronaviridae.

18. A method for disinfecting, sanitizing and/or cleaning, comprising applying an effective amount of the composition of claim 1.

19. A method for manufacturing personal care products or skincare products, comprising applying an effective amount of the composition of claim 1.

20. A disinfectant for skin or for nonliving surfaces, comprising the composition as defined in claim 1.

21. A Ganoderma lucidum strain selected from the group consisting of the strains deposited under accession numbers CBS 147377, CBS 147378, CBS 147379 and CBS 147380.

22. A method for preventing the spread of infections caused by severe acute respiratory syndrome coronavirus 2 (SARS-CoV2), comprising applying an effective amount of a mixture of biologically active compounds obtainable from any one of the isolated Ganoderma lucidum strains deposited under accession numbers CBS 147377, CBS 147378, CBS 147379, CBS 147380, or any combination thereof in preventing the spread of infections caused by severe acute respiratory syndrome coronavirus 2 (SARS-CoV2).
